(19) United States
(12) Patent Application Publication
(10) Pub. No.: US 2013/0143923 A1
(43) Pub. Date: Jun. 6, 2013

(54) CRYSTALLINE FORMS OF THALIDOMIDE AND PROCESSES FOR THEIR PREPARATION

(71) Applicants: Vinayak Govind Gore, Maharashtra (IN); Vinay Kumar Shukla, Maharashtra (IN); Madhukar Patil, Maharashtra (IN); Sandeep Mekde, Maharashtra (IN)

(72) Inventors: Vinayak Govind Gore, Maharashtra (IN); Vinay Kumar Shukla, Maharashtra (IN); Madhukar Patil, Maharashtra (IN); Sandeep Mekde, Maharashtra (IN)

(21) Appl. No.: 13/702,754

(22) PCT Filed: Jun. 9, 2011
(86) PCT No.: PCT/GB11/51078

§ 371 Date: Feb. 20, 2013

(30) Foreign Application Priority Data
Jun. 9, 2010 (IN) 1758/MUM/2010

(51) Int. Cl. A61K 31/454; C07D 401/04

(52) U.S. Cl. CPC C07D 401/04
USPC 514/323; 546/200

(57) ABSTRACT

The present invention related to crystalline forms of thalidomide having a high polymorphic purity and to processes for their preparation. The present invention also relates to pharmaceutical preparations comprising the crystalline forms for the treatment of patients suffering from autoimmune, inflammatory or angiogenic disorders.

### FIELD OF THE INVENTION

The present invention relates to crystalline forms of thalidomide having a high polymorphic purity and to processes for their preparation. The present invention also relates to pharmaceutical preparations comprising the crystalline forms for the treatment of patients suffering from autoimmune, inflammatory or angiogenic disorders.

### BACKGROUND OF THE INVENTION

Thalidomide, represented by formula (I) and chemically known as 2-(2,6-dioxo-3-piperidinyl)-1H-isoindole-1,3(2H)-dione, is a selective inhibitor of tumour necrosis factor α (TNF-α) and is useful in the treatment of erythema nodosum leprosum (ENL), a painful complication of leprosy. In addition the anti-inflammatory and immunomodulatory properties of thalidomide make it useful in the treatment of patients suffering from leukaemia, AIDS and other autoimmune diseases. Thalidomide also inhibits the growth of new blood vessels (angiogenesis), which also means it is useful in treating macular degeneration and other diseases. Thalidomide is currently marketed for the treatment of erythema nodosum leprosum (ENL). An EMEA report (EMEA/176582/2008) also outlines the use of thalidomide as a selective inhibitor of tumour necrosis factor α (TNF-α) for the treatment of patients with newly diagnosed multiple myeloma (a type of blood cancer in which immature malignant plasma cells accumulate in and eventually destroy the bone marrow).

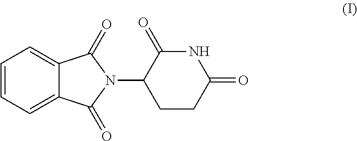 [SMILES: O=C1CCC(N2C(=O)c3ccccc3C2=O)C(=O)N1]

Thalidomide was first described by Chemie Grünenthal GmbH in GB 768821 along with a process for its preparation. The process disclosed involves cyclization of N-phthaloyl-L-glutamic acid anhydride by heating with urea or thiourea at a temperature of 170° C. to 180° C. This process suffers from poor yields and is undesirable due to the high reaction temperature and evolution of carbon dioxide and ammonia. The initial use of thalidomide was as a sedative and hypnotic.

EP 1004581 describes a process for the preparation of thalidomide by cyclization of N-phthaloyl-glutamine or N-phthaloyl-isoglutamine with N,N′-carbonyl diimidazole in dry tetrahydrofuran solvent, with heating, in the presence of an inorganic base such as sodium carbonate or sodium bicarbonate. The use of a costly ‘dry’ solvent and the use of an inorganic base which causes the formation of a heterogeneous reaction mixture, make this process not commercially viable.

CN 1405166 filed by Changchem discloses a process wherein N-phthaloyl-L-glutamine, prepared from L-glutamine and phthalic anhydride, is cyclized in 1,4-dioxane to produce thalidomide. The use of a costly solvent with significant safety requirements for the cyclization reaction makes this process undesirable on an industrial scale.

AU 2005202345 filed by Antibioticos S.P.A. discloses a ‘one pot’ synthesis for the preparation of thalidomide. As for the processes described above, agents such as phthalic anhydride or N-carbethoxyphthalimide are treated with L-glutamine to produce the intermediate N-phthaloyl-L-glutamine which, in the same vessel, is directly converted into thalidomide using a condensing agent such as thionyl chloride, carbonyl diirnidazole or phosphorous oxychloride. The process uses polar aprotic solvents such as pyridine, dimethylsulfoxide, N-methylpyrrolidone and dimethylformamide. The corrosive nature of thionyl chloride and the difficulty of the removal of high boiling point polar solvents, after reaction completion, restricts the industrial application of this process.

In WO 2009/083724, Cipla Ltd. discloses a method of preparation of thalidomide in a single reactor without isolation of any intermediates as a solid. According to the disclosure a phthaloylating agent such as phthalic acid, its esters or its derivatives (such as phthalic anhydride), phthaloyl chloride or N-carbethoxyphthalimide is treated with L-glutamine in the presence of an organic base such as a tertiary alkyl amine, e.g. triethylamine, in a non-polar organic solvent such as toluene to produce the phthaloyl derivative of L-glutamine after removal of water azeotropically. Further conversion into thalidomide is completed in the presence of a dehydrating agent such as acid anhydride, acid halide, molecular sieves or an ion exchange resin in a polar aprotic solvent, such as dimethylformamide, 1,4-dioxane, N-methylpyrrolidone, dimethylacetamide, dimethylsulfoxide etc. The product thalidomide was isolated from the reaction mixture by addition of a solvent such as a C₁ to C₄ alcohol, ketone or an ester. Azeotropic removal of water and use of corrosive dehydrating agents make the process less desirable on an industrial scale.

The patent references mentioned above all outline methods for the preparation of thalidomide. To date there are no patents or applications published which disclose methods of preparing thalidomide with selective polymorphic purity.

The existence of two polymorphic forms of racemic thalidomide is discussed in the publications J. Chem. Soc. Perkin Trans. 2, 1994, pages 2063-2067; Journal of Chemical Crystallography, 1994, vol. 24, no. 1, pages 95-99; and International Journal of Pharmaceutics, 2009, vol. 372, pages 17-23. The publications describe thalidomide in two polymorphic forms, namely α-form and β-form. The two forms are characterised in terms of their different and discrete X-ray diffraction patterns, infrared spectra and intrinsic dissolution properties. The article ‘Solid state evaluation of some thalidomide raw materials’, International Journal of Pharmaceutics, 2009, vol. 372, pages 17-23, describes the characteristics of six commercially available sources of thalidomide and concludes that there was a lack of homogeneity among the crystal habits of the samples analysed. This suggests that current processes used to produce thalidomide are not capable of producing a pure polymorph.

It is well known that physical properties such as dissolution behaviour of an API can affect its bioavailability which can affect the amount of API required in a pharmaceutical formulation. It is an aim of the formulation scientist to utilise forms of an API that provide the solid state characteristics required to provide a composition with excellent bioavailability.

Polymorphism influences every aspect of the solid state properties of an API and one of the important aspects of polymorphism in pharmaceuticals is the possibility of inter-conversion from one polymorphic form to another. It is important that pure, stable, crystalline forms are used in pharmaceutical dosage forms as conversion from a form showing greater dissolution and potentially better bioavailability to a less soluble form can potentially have disastrous consequences.

Thalidomide is a problematic drug due to its poor solubility and difficulties encountered in processing it in a tablet. It would therefore be advantageous to have a selective process whereby thalidomide can be produced with high polymorphic purity.

### OBJECT OF THE INVENTION

Accordingly, the present invention provides processes for selectively producing thalidomide in either its α-form or its β-form with high polymorphic purity.

It is a further object of the present invention to provide processes for producing thalidomide with high polymorphic purity in order to control dissolution rate in vivo, bioavailability, and further provide advantageous characteristics during dosage form manufacture, for example good conversion stability and formulation characteristics.

It is a further object of this invention to provide processes for producing thalidomide with high polymorphic purity and high chemical purity in order to minimise the presence of potentially harmful impurities and enhance the stability of the API.

### SUMMARY OF THE INVENTION

A first aspect of the present invention is a solid, anhydrous α-form of thalidomide having a polymorphic purity (as measured by XRPD or DSC, preferably as measured by XRPD) greater than or equal to 95%, preferably having a polymorphic purity greater than or equal to 97%, more preferably having a polymorphic purity greater than or equal to 99%, even more preferably having a polymorphic purity greater than or equal to 99.5%, and most preferably having a polymorphic purity greater than or equal to 99.9%.

Preferably the solid, anhydrous α-form of thalidomide according to the first aspect of the invention has a chemical purity (as measured by HPLC) greater than or equal to 99%, preferably greater than or equal to 99.5%, preferably greater than or equal to 99.8%.

Preferably the solid, anhydrous α-form of thalidomide according to the first aspect of the invention contains less than or equal to 5% of crystalline β-form of thalidomide, preferably less than or equal to 3%, preferably less than or equal to 1%, preferably less than or equal to 0.5%, preferably less than or equal to 0.1%.

A second aspect of the invention is a solid, anhydrous α-form of thalidomide having a chemical purity (as measured by HPLC) greater than or equal to 99%, preferably having a chemical purity greater than or equal to 99.5%, and most preferably having a chemical purity greater than or equal to 99.8%.

Preferably the solid, anhydrous α-form of thalidomide according to the second aspect of the invention has a polymorphic purity (as measured by XRPD or DSC, preferably as measured by XRPD) greater than or equal to 95%, preferably greater than or equal to 97%, preferably greater than or equal to 99%, preferably greater than or equal to 99.5%, preferably greater than or equal to 99.9%.

Preferably the solid, anhydrous α-form of thalidomide according to the second aspect of the invention contains less than or equal to 5% of crystalline β-form of thalidomide, preferably less than or equal to 3%, preferably less than or equal to 1%, preferably less than or equal to 0.5%, preferably less than or equal to 0.1%.

A third aspect of the present invention is a process for preparing a solid, anhydrous, crystalline α-form of thalidomide comprising cyclizing N-phthaloyl-glutamine in an organic solvent system and isolating the solid, anhydrous, crystalline α-form of thalidomide.

The N-phthaloyl-glutamine may be N-phthaloyl-L-glutamine or N-phthaloyl-D-glutamine or a mixture thereof, such as racemic N-phthaloyl-DL-glutamine. Preferably the N-phthaloyl-glutamine is N-phthaloyl-L-glutamine.

Preferably the N-phthaloyl-glutamine is cyclized by reaction with a coupling agent.

Preferably the coupling agent is selected from the group consisting of carbonyl diimidazole (CDI), phosphorus oxychloride, thionyl chloride, urea, thiourea, acid chloride, acetic anhydride, phosgene, ethyl chloroformate, thionyl diimidazole, pivaloyl chloride, tosyl chloride, mesyl chloride, tosyl imidazole, 1-ethyl-3-(3-dimethylaminopropyl) carbodiimide (EDCI), 2-chloro-N-methyl-pyridinium iodide, 2-(1H-benzotriazol-1-yl)-1,1,3,3-tetramethyl uronium hexafluorophosphate (HBTU) and 2-(benzotriazol-1-yl)oxytris(dimethylamino) phosphonium hexafluorophosphate (BOP) or mixtures thereof. In one embodiment, the coupling agent is not an acid anhydride or an acid halide. Most preferably the coupling agent is carbonyl diimidazole (CDI).

Preferably the N-phthaloyl-glutamine is cyclized in the presence of a catalyst.

Preferably the catalyst is an organic base. Preferably the catalyst is selected from the group consisting of 4-dimethylaminopyridine (DMAP), pyridine, diethylaminopyridine, 1,8-diazabicyclo[5,4,0]undec-7-ene (DBU), 1,4-diazabicyclo[2,2,2]octane (DABCO) and 1,5-diazabicyclo[4,3,0]non-5-ene (DBN) or mixtures thereof. Most preferably the catalyst is 4-dimethylaminopyridine (DMAP).

Preferably the organic solvent system comprises solvents selected from the group comprising straight chain or branched aliphatic ketones, aliphatic nitriles, ethers or mixtures thereof.

Preferably the straight chain or branched aliphatic ketone is selected from the group consisting of acetone and butanone or mixtures thereof. Most preferably the straight chain or branched aliphatic ketone is acetone.

Preferably the aliphatic nitrile is selected from the group consisting of acetonitrile and propionitrile or mixtures thereof. Most preferably the aliphatic nitrile is acetonitrile.

Preferably the ether is selected from the group consisting of tetrahydrofuran (THF) and tertiary butyl methyl ether (TBME) or mixtures thereof. Preferably the ether is a mixture of two or more ethers. Most preferably the ether is a mixture of tetrahydrofuran (THF) and tertiary butyl methyl ether (TBME).

In one embodiment the ether is not 2-ethoxy-ethanol. In another embodiment the ether is not anhydrous THF.

Preferably the reaction mixture is heated to a temperature between about 50° C. and about 100° C., most preferably heated to a temperature between about 50° C. and about 77° C.

Preferably the reaction mixture is further cooled in order to isolate the solid, anhydrous, crystalline α-form of thalidomide. Most preferably the reaction mixture is cooled to a temperature between about 25° C. and about 30° C.

A fourth aspect of the present invention is a solid, anhydrous β-form of thalidomide having a polymorphic purity (as measured by XRPD or DSC, preferably as measured by XRPD) greater than or equal to 95%, preferably having a polymorphic purity greater than or equal to 97%, more preferably having a polymorphic purity greater than or equal to 99%, even more preferably having a polymorphic purity greater than or equal to 99.5%, and most preferably having a polymorphic purity greater than or equal to 99.9%.

Preferably the solid, anhydrous β-form of thalidomide according to the fourth aspect of the invention has a chemical purity (as measured by HPLC) greater than or equal to 99%, preferably greater than or equal to 99.5%, preferably greater than or equal to 99.8%.

Preferably the solid, anhydrous β-form of thalidomide according to the fourth aspect of the invention contains less than or equal to 5% of crystalline α-form of thalidomide, preferably less than or equal to 3%, preferably less than or equal to 1%, preferably less than or equal to 0.5%, preferably less than or equal to 0.1%.

A fifth aspect of the invention is a solid, anhydrous β-form of thalidomide having a chemical purity (as measured by HPLC) greater than or equal to 99%, preferably having a chemical purity greater than or equal to 99.5%, and most preferably having a chemical purity greater than or equal to 99.8%.

Preferably the solid, anhydrous β-form of thalidomide according to the fifth aspect of the invention has a polymorphic purity (as measured by XRPD or DSC, preferably as measured by XRPD) greater than or equal to 95%, preferably greater than or equal to 97%, preferably greater than or equal to 99%, preferably greater than or equal to 99.5%, preferably greater than or equal to 99.9%

Preferably the solid, anhydrous p-form of thalidomide according to the fifth aspect of the invention contains less than or equal to 5% of crystalline a-form of thalidomide, preferably less than or equal to 3%, preferably less than or equal to 1%, preferably less than or equal to 0.5%, preferably less than or equal to 0.1%.

A sixth aspect of the present invention is a process for preparing a solid, anhydrous, crystalline β-form of thalidomide comprising cyclizing N-phthaloyl-glutamine in an organic solvent system, heating the reaction mixture and isolating the solid, anhydrous, crystalline β-form of thalidomide.

The N-phthaloyl-glutamine may be N-phthaloyl-L-glutamine or N-phthaloyl-D-glutamine or a mixture thereof, such as racemic N-phthaloyl-DL-glutamine. Preferably the N-phthaloyl-glutamine is N-phthaloyl-L-glutamine

Preferably the N-phthaloyl-glutamine is cyclized by reaction with a coupling agent.

Preferably the coupling agent is selected from the group consisting of carbonyl diimidazole (CDI), phosphorus oxychloride, thionyl chloride, urea, thiourea, acid chloride, acetic anhydride, phosgene, ethyl chloroformate, thionyl diimidazole, pivaloyl chloride, tosyl chloride, mesyl chloride, tosyl imidazole, 1-ethyl-3-(3-dimethylaminopropyl) carbodiimide (EDCI), 2-chloro-N-methyl-pyridinium iodide, 2-(1H-benzotriazol-1-yl)-1,1,3,3-tetramethyl uronium hexafluorophosphate (HBTU) and 2-(benzotriazol-1-yl)oxytris(dimethylamino) phosphonium hexafluorophosphate (BOP) or mixtures thereof. In one embodiment, the coupling agent is not an acid anhydride or an acid halide. Most preferably the coupling agent is carbonyl diimidazole (CDI).

Preferably the N-phthaloyl-glutamine is cyclized in the presence of a catalyst.

Preferably the catalyst is an organic base. Preferably the catalyst is selected from the group consisting of 4-dimethylaminopyridine (DMAP), pyridine, diethylaminopyridine, 1,8-diazabicyclo[5,4,0]undec-7-ene (DBU), 1,4-diazabicyclo[2,2,2]octane (DABCO) and 1,5-diazabicydo[4,3,0]non-5-ene (DBN) or mixtures thereof. Most preferably the catalyst is 4-dimethylaminopyridine (DMAP).

Preferably the organic solvent system comprises solvents selected from the group comprising dimethylformamide (DMF), dimethylacetamide or mixtures thereof. Most preferably the solvent is dimethylformamide (DMF).

Preferably the reaction mixture is heated to a temperature between about 50° C. and about 100° C. Most preferably the reaction mixture is heated to a temperature between about 70° C. and about 75° C.

Preferably isolating the solid, anhydrous, crystalline β-form of thalidomide comprises removal of the organic solvent system, addition of a second solvent preferably selected from the group consisting of methanol, water, acetone or mixtures thereof, and isolating the solid, anhydrous, crystalline β-form of thalidomide.

Preferably the second solvent is selected from the group consisting of acetone and a mixture of methanol and water.

A seventh aspect of the present invention is an anhydrous, crystalline α-form of thalidomide containing less than or equal to 5% of crystalline β-form of thalidomide, preferably less than or equal to 3%, preferably less than or equal to 1%, preferably less than or equal to 0.5%, preferably less than or equal to 0.1%.

Preferably the anhydrous, crystalline α-form of thalidomide according to the seventh aspect of the invention has a chemical purity (as measured by HPLC) greater than or equal to 99%, preferably greater than or equal to 99.5%, preferably greater than or equal to 99.8%.

Preferably the anhydrous, crystalline α-form of thalidomide according to the seventh aspect of the invention has a polymorphic purity (as measured by XRPD or DSC, preferably as measured by XRPD) greater than or equal to 95%, preferably greater than or equal to 97%, preferably greater than or equal to 99%, preferably greater than or equal to 99.5%, preferably greater than or equal to 99.9%.

An eighth aspect of the present invention is an anhydrous, crystalline β-form of thalidomide containing less than or equal to 5% of crystalline α-form of thalidomide, preferably less than or equal to 3%, preferably less than or equal to 1%, preferably less than or equal to 0.5%, preferably less than or equal to 0.1%.

Preferably the anhydrous, crystalline β-form of thalidomide according to the eighth aspect of the invention has a chemical purity (as measured by HPLC) greater than or equal to 99%, preferably greater than or equal to 99.5%, preferably greater than or equal to 99.8%.

Preferably the anhydrous, crystalline β-form of thalidomide according to the eighth aspect of the invention has a polymorphic purity (as measured by XRPD or DSC, preferably as measured by XRPD) greater than or equal to 95%, preferably greater than or equal to 97%, preferably greater than or equal to 99%, preferably greater than or equal to 99.5%, preferably greater than or equal to 99.9%.

A ninth aspect of the present invention is a process for preparing a pure, anhydrous, crystalline α-form of thalidomide comprising dissolving thalidomide in dimethylsulfoxide (DMSO), adding the mixture to methanol containing suspended seed crystals of the α-form of thalidomide, and isolating the pure, anhydrous, crystalline α-form of thalidomide.

Preferably the thalidomide starting material is selected from the group consisting of crystalline α-form of thalidomide and a mixture of α-form and β-form.

Preferably the reaction mixture is heated to a temperature between about 40° C. and about 50° C.

Preferably the reaction mixture is further cooled in order to isolate the pure, anhydrous, crystalline α-form of thalidomide. Most preferably the reaction mixture is cooled to a temperature between about 30° C. and about 40° C.

A tenth aspect of the present invention is a pure, anhydrous, crystalline α-form of thalidomide having a chemical purity (as measured by HPLC) greater than or equal to 99.9%, prepared by a process according to the ninth aspect of the present invention.

An eleventh aspect of the present invention is a process for preparing a pure, anhydrous, crystalline β-form of thalidomide comprising dissolving thalidomide in dimethylformamide (DMF), heating the reaction mixture, and isolating the pure, anhydrous, crystalline β-form of thalidomide.

Preferably the thalidomide starting material is selected from the group consisting of crystalline α-form of thalidomide, crystalline β-form of thalidomide and a mixture of α-form and β-form.

Preferably the reaction mixture is heated to a temperature between about 50° C. and about 100° C. Most preferably the reaction mixture is heated to a temperature between about 70° C. and about 75° C.

Preferably isolating the pure, anhydrous, crystalline β-form of thalidomide comprises removal of DMF, addition of a second solvent preferably selected from the group consisting of methanol, water, acetone or mixtures thereof, and isolating the pure, anhydrous, crystalline β-form of thalidomide.

Preferably the second solvent is selected from the group consisting of acetone and a mixture of methanol and water.

A twelfth aspect of the present invention is a pure, anhydrous, crystalline β-form of thalidomide having a chemical purity (as measured by HPLC) greater than or equal to 99.9%, prepared by a process according to the eleventh aspect of the present invention.

In any of the processes of the present invention, preferably the anhydrous, crystalline α-form or β-form of thalidomide is prepared either from N-phthaloyl-glutamine in a molar yield of 50% or more, preferably 60% or more, preferably 70% or more, preferably 80% or more, or from thalidomide in a molar yield of 50% or more, preferably 60% or more, preferably 70% or more, preferably 80% or more, preferably 90% or more, preferably 95% or more.

In any of the processes of the present invention, preferably the anhydrous, crystalline α-form or β-form of thalidomide is prepared on an industrial scale, preferably in batches of 100 g or more, preferably 250 g or more, preferably 500 g or more, preferably 1 kg or more, preferably 5 kg or more, preferably 10 kg or more, preferably 25 kg or more.

Preferably the anhydrous, crystalline α-form of thalidomide according to the first, second, seventh or tenth aspect of the invention or prepared by a process according to the third or ninth aspect of the invention is suitable for use in medicine, preferably suitable for treating an autoimmune, inflammatory or angiogenic disorder, preferably suitable for treating erythema nodosum leprosum (ENL) and multiple myeloma.

Preferably the anhydrous, crystalline β-form of thalidomide according to the fourth, fifth, eighth or twelfth aspect of the invention or prepared by a process according to the sixth or eleventh aspect of the invention is suitable for use in medicine, preferably suitable for treating an autoimmune, inflammatory or angiogenic disorder, preferably suitable for treating erythema nodosum leprosum (ENL) and multiple myeloma.

A thirteenth aspect of the present invention is a pharmaceutical composition comprising an anhydrous, crystalline α-form of thalidomide according to the first, second, seventh or tenth aspect of the invention or prepared by a process according to the third or ninth aspect of the invention, and one or more pharmaceutically acceptable excipients.

A fourteenth aspect of the present invention is a pharmaceutical composition comprising an anhydrous, crystalline β-form of thalidomide according to the fourth, fifth, eighth or twelfth aspect of the invention or prepared by a process according to the sixth or eleventh aspect of the invention, and one or more pharmaceutically acceptable excipients.

A fifteenth aspect of the present invention is the use of the anhydrous, crystalline α-form of thalidomide according to the first, second, seventh or tenth aspect of the invention or prepared by a process according to the third or ninth aspect of the invention, or the use of the anhydrous, crystalline β-form of thalidomide according to the fourth, fifth, eighth or twelfth aspect of the invention or prepared by a process according to the sixth or eleventh aspect of the invention, or the use of a pharmaceutical composition according to the thirteenth or fourteenth aspect of the present invention, in the manufacture of a medicament for the treatment of erythema nodosum leprosum (ENL).

A sixteenth aspect of the present invention is the use of the anhydrous, crystalline α-form of thalidomide according to the first, second, seventh or tenth aspect of the invention or prepared by a process according to the third or ninth aspect of the invention, or the use of the anhydrous, crystalline β-form of thalidomide according to the fourth, fifth, eighth or twelfth aspect of the invention or prepared by a process according to the sixth or eleventh aspect of the invention, or the use of a pharmaceutical composition according to the thirteenth or fourteenth aspect of the present invention, in the manufacture of a medicament for the treatment of multiple myeloma.

A seventeenth aspect of the present invention is a method of treating erythema nodosum leprosum (ENL), comprising administering to a patient in need thereof a therapeutically effective amount of the anhydrous, crystalline α-form of thalidomide according to the first, second, seventh or tenth aspect of the invention or prepared by a process according to the third or ninth aspect of the invention, or a therapeutically effective amount of the anhydrous, crystalline β-form of thalidomide according to the fourth, fifth, eighth or twelfth aspect of the invention or prepared by a process according to the sixth or eleventh aspect of the invention, or a therapeutically effective amount of a pharmaceutical composition according to the thirteenth or fourteenth aspect of the present invention. Preferably the patient is a mammal, preferably a human.

An eighteenth aspect of the present invention is a method of treating multiple myeloma, comprising administering to a patient in need thereof a therapeutically effective amount of the anhydrous, crystalline α-form of thalidomide according to the first, second, seventh or tenth aspect of the invention or prepared by a process according to the third or ninth aspect of the invention, or a therapeutically effective amount of the anhydrous, crystalline β-form of thalidomide according to the fourth, fifth, eighth or twelfth aspect of the invention or prepared by a process according to the sixth or eleventh aspect of the invention, or a therapeutically effective amount of a pharmaceutical composition according to the thirteenth or fourteenth aspect of the present invention. Preferably the patient is a mammal, preferably a human.

### BRIEF DESCRIPTION OF THE ACCOMPANYING FIGURES

FIG. 1: Synthesis scheme of thalidomide following a preferred process according to the invention.

FIG. 2: XRPD trace of a pure, anhydrous, crystalline α-form of thalidomide according to the invention.

FIG. 3: XRPD trace of a pure, anhydrous, crystalline β-form of thalidomide according to the invention.

FIG. 4: Differential Scanning Calorimetry of an anhydrous, crystalline α-form of thalidomide according to the invention.

FIG. 5: Differential Scanning Calorimetry of an anhydrous, crystalline β-form of thalidomide according to the invention.

FIG. 6: FTIR spectrum of an anhydrous, crystalline α-form of thalidomide according to the invention.

FIG. 7: FTIR spectrum of an anhydrous, crystalline β-form of thalidomide according to the invention.

### DETAILED DESCRIPTION OF THE INVENTION

As outlined above, the present invention provides polymorphically pure, stable, anhydrous α-form and anhydrous β-form of thalidomide which have beneficial properties and which avoid the problems associated with the polymorphic mixtures produced by the prior art processes.

Preferred embodiments of the pure polymorphs are described below.

Both the anhydrous α-form and anhydrous β-form of the present invention have a polymorphic purity of greater than or equal to 95%, preferably having a polymorphic purity of greater than or equal to 97%, more preferably having a polymorphic purity of greater than or equal to 99%, even more preferably having a polymorphic purity of greater than or equal to 99.5%, and most preferably having a polymorphic purity of greater than or equal to 99.9%.

These two forms were characterised by the inventors by differential scanning calorimetry (DSC), X-ray diffraction (XRPD) and Fourier transform infrared spectroscopy (FTIR). Additional polymorphic purity analysis of the individual polymorphs was completed by XRPD methods.

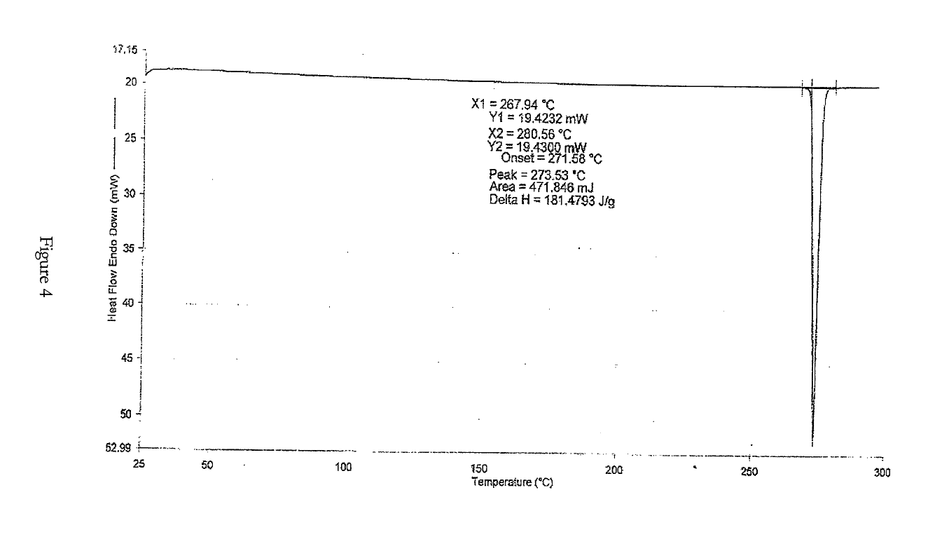

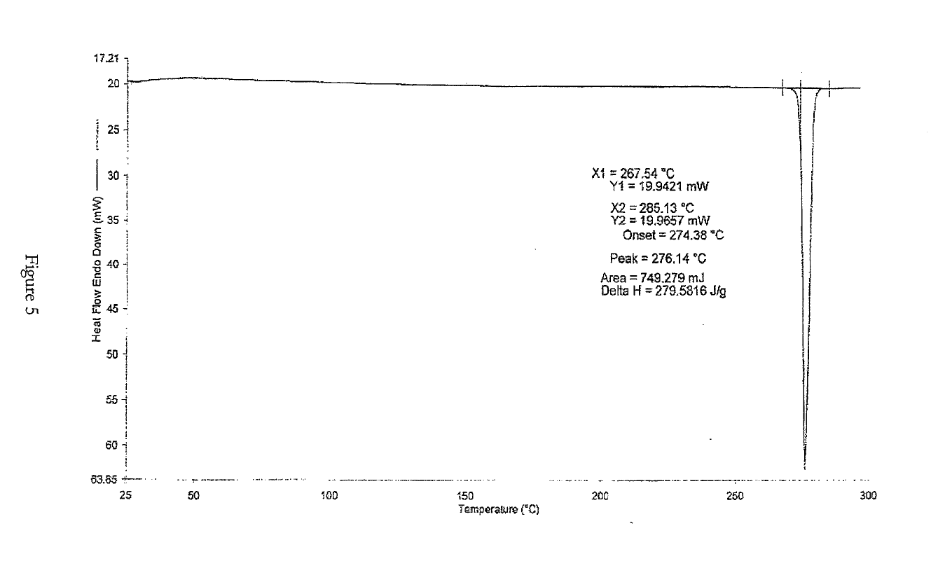

During development the inventors found that DSC was an indicative analysis method for determining the polymorphic form of thalidomide, with the anhydrous α-form giving a single endothermic peak between 273° C. and 275° C. and the anhydrous β-form giving a single endothermic peak between 276° C. and 280° C. DSC thermograms indicative of the forms of the present invention are presented in FIGS. 4 and 5.

The DSC thermograms were recorded on a Perkin Elmer Pyris 6 instrument over a range of 25° C. to 350° C. at a heating rate of 10° C/min. Samples were prepared in a sealed pan pierced immediately prior to analysis.

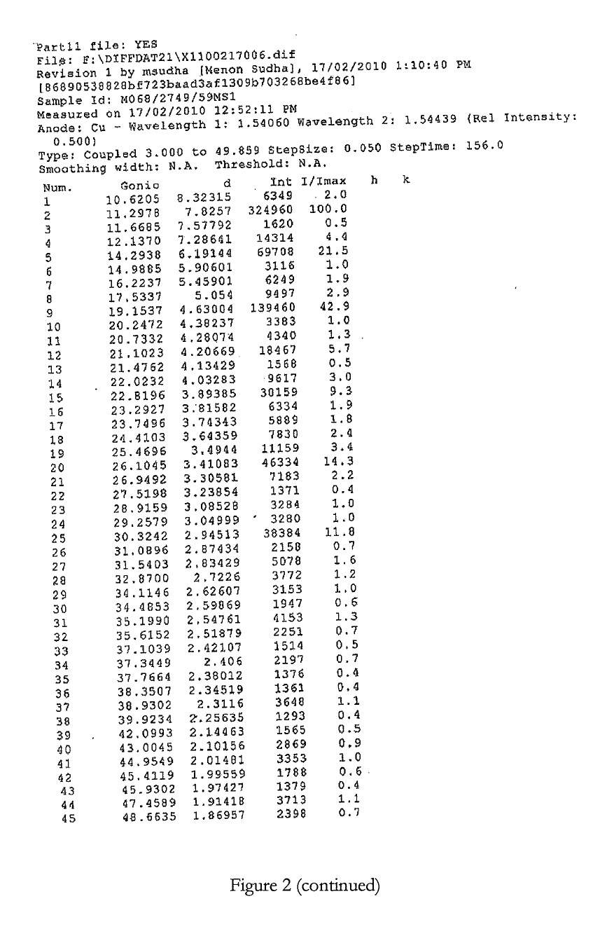

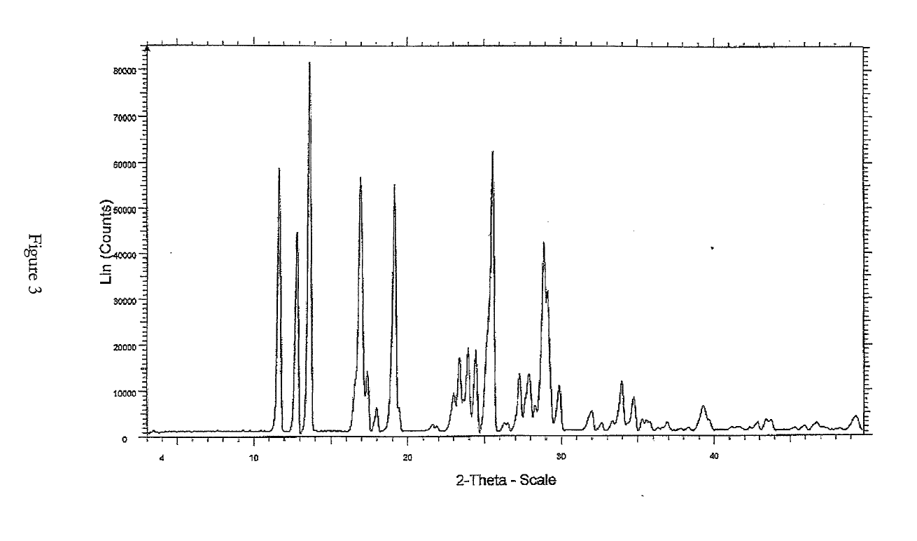

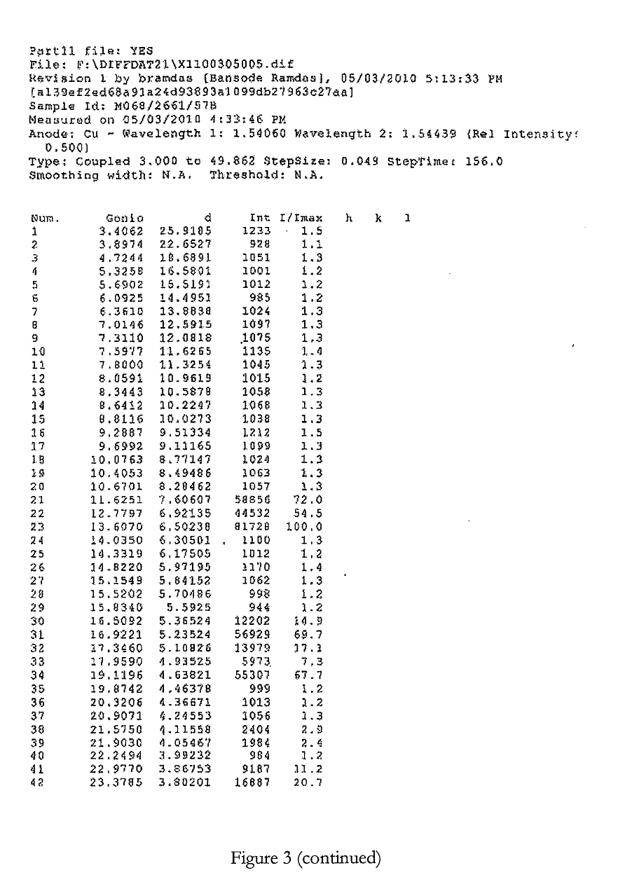

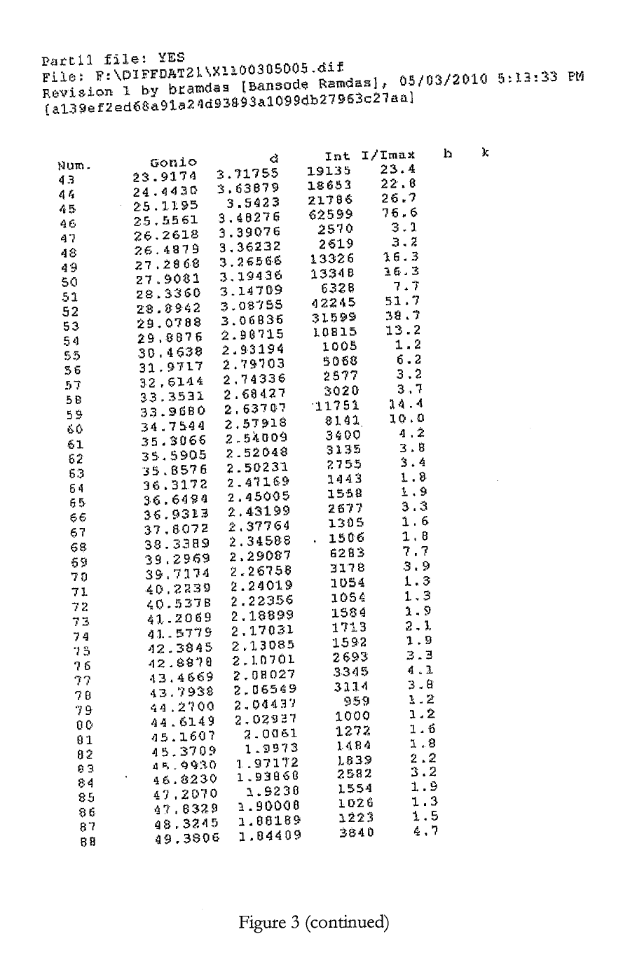

The inventors found that XRPD is also a distinctive technique for the measurement of the anhydrous α-form and the anhydrous β-form of thalidomide. X-ray diffractograms of the forms of the present invention are presented in FIGS. 2 and 3.

The X-ray diffractogram of the anhydrous α-form of thalidomide contains characteristic peaks at about 11.30, 14.30, 19.20, 22.8, 26.1 and 30.40±0.2 °2-theta or the X-ray diffractogram of the anhydrous α-form of thalidomide contains characteristic peaks at about 11.30, 14.29, 19.15, 22.82, 26.10 and 30.32±0.2 °2-theta.

The X-ray diffractogram of the anhydrous β-form of thalidomide contains characteristic peaks at about 11.78, 12.96, 13.75, 17.06, 19.26, 24.06, 25.73, 29.05 and 29.29±0.2 °2-theta or the X-ray diffractogram of the anhydrous β-form of thalidomide contains characteristic peaks at about 11.63, 12.78, 13.61, 16.92, 19.12, 23.92, 25.12, 25.56, 28.89 and 29.08±0.2 °2-theta.

XRPD analyses were carried out on a Bruker D8 Advance diffractometer using a Cu Kα1 source. The diffractograms were collected over an angular range of 3° to 50° 2-theta in steps of 0.05° 2-theta with a measurement time of 156 seconds per step.

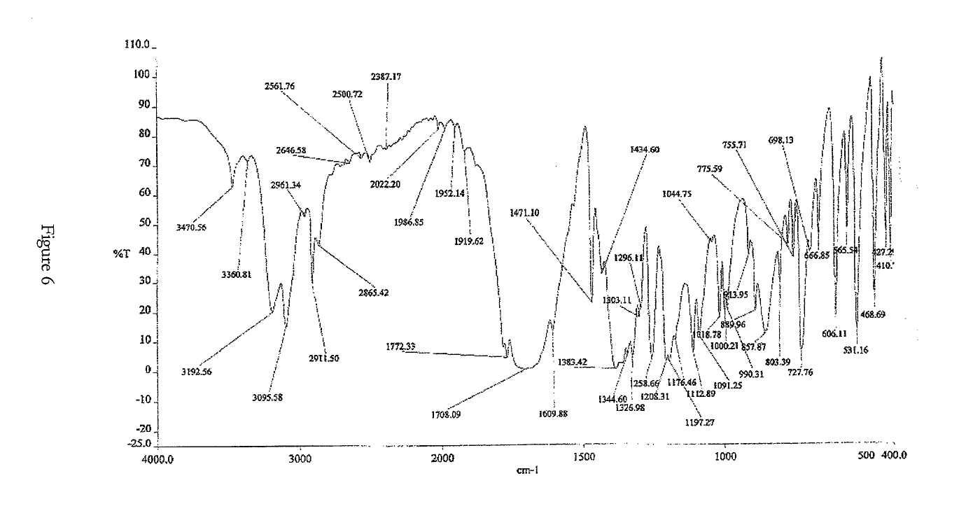

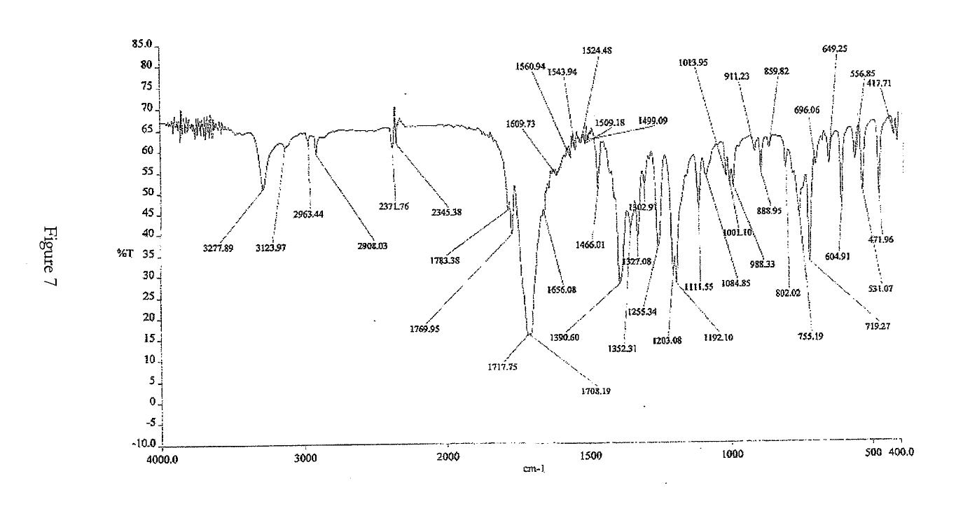

Additionally FTIR was found to be indicative of the polymorphic forms with spectra indicative of the forms of the present invention presented in FIGS. 6 and 7.

The FTIR spectrum of the anhydrous α-form contains characteristic absorption bands at 3196, 3098 and 859 cm⁻¹, which were found to be absent in the spectrum of the anhydrous β-form. The FTIR spectrum of the anhydrous β-form contains characteristic absorption bands at 3277 and 755 cm⁻¹, which were not found in the spectrum of the anhydrous α-form.

The FTIR spectra were recorded on a Perkin Elmer Spectrum BX II spectrophotometer over the range of 400 to 4000 cm⁻¹. The IR spectra were obtained from samples prepared as dispersion in potassium bromide pressed into a disc.

Chemical purity was measured by reversed phase high performance liquid chromatography (HPLC). The HPLC purity results were collected using a Waters E-2695 HPLC system with a Waters W 2487 UV detector at a wavelength of 218 nm, with separation carried out using a L1, C-18 Reversed Phase column.

In addition, processes have been developed to selectively prepare the anhydrous α-form and the anhydrous β-form of thalidomide which give the selected form with a high polymorphic and chemical purity.

Preferred embodiments of the present invention are described below.

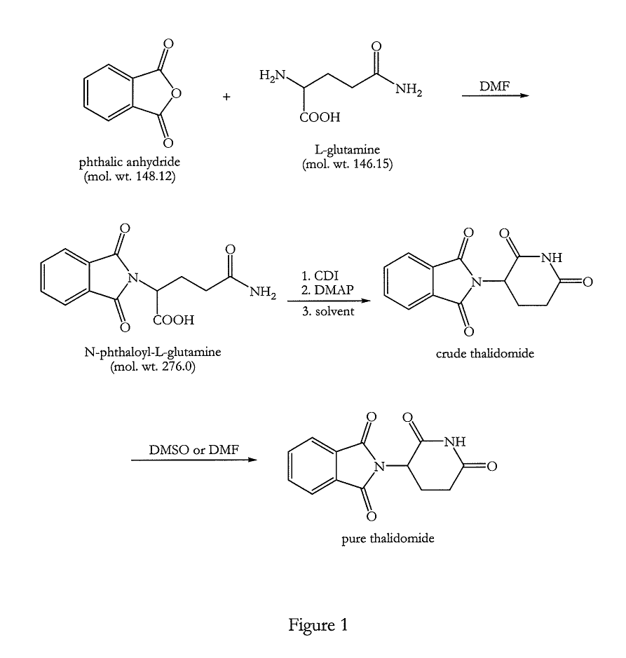

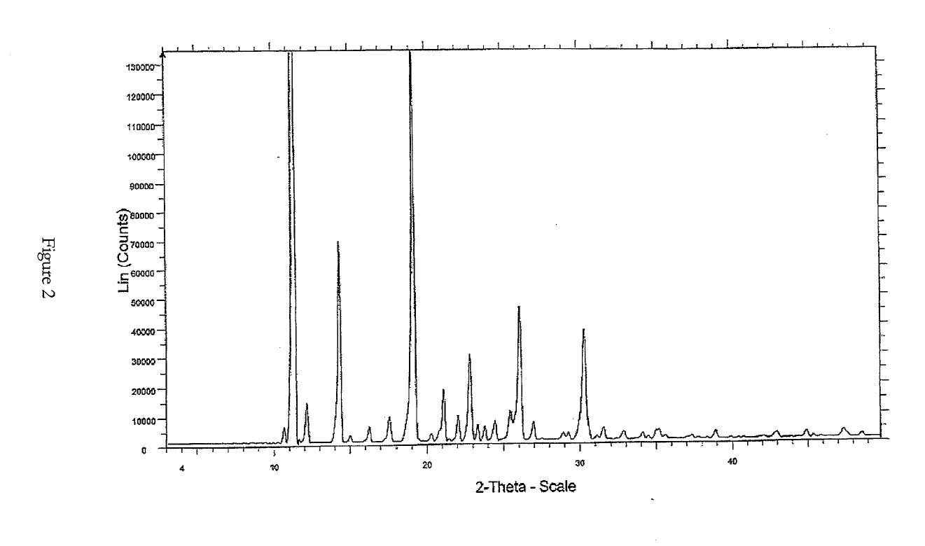

A preferred process for the preparation of thalidomide of the present invention is outlined in FIG. 1 and comprises the reaction of phthalic anhydride with L-glutamine in dimethylformamide (DMF) to give N-phthaloyl-L-glutamine. The N-phthaloyl-L-glutamine is then preferably reacted with a coupling agent, preferably N,N′-carbonyl dimidazole (CDI), preferably in the presence of a catalyst, preferably a catalytic amount of 4-dimethylaminopyridine (DMAP), to complete the cyclization to give thalidomide.

A preferred process for the preparation of the anhydrous α-form of thalidomide comprises reacting the starting material N-phthaloyl-L-glutamine with a cyclization agent such as carbonyl diimidazole, in the presence of a catalytic amount of 4-dimethylaminopyridine, in an organic solvent system, followed by isolating the solid, anhydrous, crystalline α-form of thalidomide.

The inventors have found that it is advantageous if the reaction mixture is heated to a temperature between 30° C. and 100° C. However it was found that it is most advantageous to heat the reaction mixture to a temperature between 50° C. and 77° C. The duration of heating required was found to be a period of between 2 and 8 hours, most preferably a period of between 5 and 8 hours.

It has also been found that it is advantageous to cool the reaction mixture to a temperature between 5° C. and 30° C. in order to isolate the solid, anhydrous, crystalline α-form of thalidomide. It was found to be most advantageous to cool the reaction mixture to a temperature between 25° C. and 30° C.

Another preferred embodiment of the present invention provides a process for preparing a pure, anhydrous, crystalline α-form of thalidomide from a starting material selected from the group consisting of crystalline α-form of thalidomide and a mixture of α-form and β-form, comprising dissolving the starting material in dimethylsulfoxide (DMSO), adding the mixture to methanol containing suspended seed crystals of α-form, and isolating pure, solid, anhydrous, crystalline α-form of thalidomide. The inventors have found that it is advantageous to heat the reaction mixture to a temperature between 30° C. and 80° C., however it is most advantageous to heat the reaction mixture to a temperature between 40° C. and 50° C.

It has also been found that it is advantageous to cool the reaction mixture to a temperature between 5° C. and 40° C. in order to isolate the solid, anhydrous, crystalline α-form of thalidomide. It was found to be most advantageous to cool the reaction mixture to a temperature between 35° C. and 40° C.

In a preferred embodiment of the processes to prepare the anhydrous α-form of thalidomide, the isolation of the resultant anhydrous, crystalline α-form is completed by filtration, followed by washing of the isolated solid with a C₁ to C₄ aliphatic alcohol selected from the group consisting of methanol, ethanol, 1-propanol, 2-propanol, 1-butanol, and 2-butanol, most preferably methanol.

A preferred process for the preparation of the anhydrous β-form of thalidomide comprises reacting the starting material N-phthaloyl-L-glutamine with a cyclization agent such as carbonyl diimidazole, in the presence of a catalytic amount of 4-dimethylaminopyridine, in an organic solvent system, heating the reaction mixture to a temperature between about 50° C. and about 100° C., most preferably between about 70° C. and about 75° C., and isolating the anhydrous, crystalline β-form of thalidomide. Preferably isolating the solid, anhydrous, crystalline β-form of thalidomide comprises removal of the organic solvent system by distillation under reduced pressure, addition of a second solvent selected from the group consisting of methanol, water, acetone and mixtures thereof, and isolating the anhydrous, crystalline β-form of thalidomide.

Another preferred embodiment of the present invention provides a process for preparing a pure, anhydrous, crystalline β-form of thalidomide from a starting material selected from the group consisting of crystalline α-form of thalidomide, crystalline β-form of thalidomide and a mixture of α-form and β-form, comprising dissolving the starting material in dimethylformamide (DMF), heating the reaction mixture to a temperature between about 50° C. and about 100° C., most preferably between about 70° C. and about 75° C., and isolating the anhydrous, crystalline β-form of thalidomide. Preferably isolating the solid, anhydrous, crystalline β-form of thalidomide comprises removal of DMF by distillation under reduced pressure, addition of a second solvent selected from the group consisting of methanol, water, acetone and mixtures thereof, and isolating the anhydrous, crystalline β-form of thalidomide.

In a preferred embodiment of the processes to prepare the anhydrous β-form of thalidomide, the isolation of the resultant anhydrous, crystalline β-form is completed by filtration, followed by washing of the isolated solid with a solvent preferably selected from the group consisting of methanol, water, acetone and mixtures thereof.

In preferred embodiments of all of the processes of the present invention, the final stage of extraction of the anhydrous crystalline form (either α-form or β-form) involves drying of the filtered and washed solid to a constant weight. Preferably the drying is carried out under reduced pressure (˜100 mmHg) at a temperature between 40° C. and 70° C. and most preferably between 50° C. and 60° C.

Another preferred embodiment of the present invention is a pharmaceutical formulation containing the anhydrous α-form or anhydrous β-form of thalidomide of the present invention.

Yet another preferred embodiment of the present invention is the use of the pharmaceutical formulations outlined above for the treatment of erythema nodosum leprosum (ENL) (a painful complication of leprosy) and in the treatment of multiple myeloma (a type of blood cancer in which immature malignant plasma cells accumulate in and eventually destroy the bone marrow). In the treatment of multiple myeloma thalidomide of the present invention may be used alone or in combination with other therapeutic agents, such as steroids (including, but not limited to, dexamethasone, hydrocortisone, cortisone acetate, prednisone, methylprednisolone, betamethasone, triamcinolone, beclomethasone, fludrocortisone acetate, deoxycorticosterone acetate (DOCA) and aldosterone) and other chemotherapeutic agents useful in the treatment of cancer (including, but not limited to, lenalidomide, melphalan and bortezomib). Some preferred combinations include: thalidomide in combination with dexamethasone and thalidomide in combination melphalan and prednisone.

In addition to the active ingredient(s), the pharmaceutical compositions of the present invention may contain one or more excipients. Excipients are added to the composition for a variety of purposes. Diluents increase the bulk of a solid pharmaceutical composition and may make a pharmaceutical dosage form containing the composition easier for the patient and care giver to handle. Diluents for solid compositions include, for example, microcrystalline cellulose (e.g. Avicel®), microtine cellulose, lactose, starch, pregelatinized starch, calcium carbonate, calcium sulphate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. Eudragit®), potassium chloride, powdered cellulose, sodium chloride, sorbitol and talc.

Solid pharmaceutical compositions that are compacted into a dosage form, such as a tablet, may include excipients whose functions include helping to bind the active ingredient and other excipients together after compression. Binders for solid pharmaceutical compositions include acacia, alginic acid, carbomer (e.g. Carbopol®), carboxymethyl cellulose sodium, dextrin, ethyl cellulose, gelatin, guar gum, hydrogenated vegetable oil, hydroxyethyl cellulose, hydroxypropyl cellulose (e.g. Klucel®), hydroxypropyl methyl cellulose (e.g. Methocel®), liquid glucose, magnesium aluminium silicate, maltodextrin, methyl cellulose, polymethacrylates, povidone (e.g. Kollidon®, Plasdone®), pregelatinized starch, sodium alginate and starch.

The dissolution rate of a compacted solid pharmaceutical composition in the patient's stomach may be increased by the addition of a disintegrant to the composition. Disintegrants include alginic acid, carboxymethyl cellulose calcium, carboxymethyl cellulose sodium (e.g. Ac-Di-Sol®, Primellose®), colloidal silicon dioxide, croscarmellose sodium, crospovidone (e.g. Kollidon®, Polyplasdone®), guar gum, magnesium aluminium silicate, methyl cellulose, microcrystalline cellulose, polacrilin potassium, powdered cellulose, pregelatinized starch, sodium alginate, sodium starch glycolate (e.g. Explotab®) and starch.

Glidants can be added to improve the flowability of a non-compacted solid composition and to improve the accuracy of dosing. Excipients that may function as glidants include colloidal silicon dioxide, magnesium trisilicate, powdered cellulose, starch, talc and tribasic calcium phosphate.

When a dosage form such as a tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc and zinc stearate.

Flavouring agents and flavour enhancers make the dosage form more palatable to the patient. Common flavouring agents and flavour enhancers for pharmaceutical products that may be included in the composition of the present invention include maltol, vanillin, ethyl vanillin, menthol, citric acid, fumaric acid, ethyl maltol and tartaric acid.

Solid and liquid compositions may also be dyed using any pharmaceutically acceptable colorant to improve their appearance and/or facilitate patient identification of the product and unit dosage level.

In liquid pharmaceutical compositions of the present invention, the crystalline α- or β-form of thalidomide according to the invention and any other solid excipients are dissolved or suspended in a liquid carrier such as water, vegetable oil, alcohol, polyethylene glycol, propylene glycol or glycerine.

Liquid pharmaceutical compositions may further contain emulsifying agents to disperse uniformly throughout the composition an active ingredient or other excipient that is not soluble in the liquid carrier. Emulsifying agents that may be useful in liquid compositions of the present invention include, for example, gelatin, egg yolk, casein, cholesterol, acacia, tragacanth, chondrus, pectin, methyl cellulose, carbomer, cetostearyl alcohol and cetyl alcohol.

Liquid pharmaceutical compositions of the present invention may also contain a viscosity enhancing agent to improve the mouth-feel or organoleptic qualities of the product and/or coat the lining of the gastrointestinal tract. Such agents include acacia, alginic acid, bentonite, carbomer, carboxymethyl cellulose calcium or sodium, cetostearyl alcohol, methyl cellulose, ethyl cellulose, gelatin, guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth and xanthan gum.

Sweetening agents such as sorbitol, saccharin, sodium saccharin, sucrose, aspartame, fructose, mannitol and invert sugar may be added to improve the taste.

Preservatives and chelating agents such as alcohol, sodium benzoate, butylated hydroxytoluene, butylated hydroxyanisole and ethylenediaminetetraacetic acid may be added at levels safe for ingestion to improve storage stability.

According to the present invention, a liquid composition may also contain a buffet such as gluconic acid, lactic acid, citric acid or acetic acid, sodium gluconate, sodium lactate, sodium citrate or sodium acetate.

Selection of excipients and the amounts used may be readily determined by the formulation scientist based upon experience and consideration of standard procedures and reference works in the field.

The solid compositions of the present invention include powders, granulates, aggregates and compacted compositions. The dosages include dosages suitable for oral, buccal, rectal, parenteral (including subcutaneous, intramuscular and intravenous), inhalant and ophthalmic administration. Although the most suitable administration in any given case will depend on the nature and severity of the condition being treated, the most preferred route of the present invention is oral. The dosages may be conveniently presented in unit dosage form and prepared by any of the methods well-known in the pharmaceutical arts. Dosage forms include solid dosage forms like tablets, powders, capsules, suppositories, sachets, troches and lozenges, as well as liquid syrups, suspensions and elixirs.

The dosage form of the present invention may be a capsule containing the composition, preferably a powdered or granulated solid composition of the invention, within either a hard or a soft shell. The shell may be made from gelatin and optionally contain a plasticizer such as glycerine and sorbitol, and an opacifying agent or colourant. The active ingredient and excipients may be formulated into compositions and dosage forms according to methods known in the art.

A composition for tabletting or capsule filling may be prepared by wet granulation. In wet granulation, some or all of the active ingredient and excipients in powder form are blended and then further mixed in the presence of a liquid, typically water, that causes the powders to clump into granules. The granulate is screened and/or milled, dried and then screened and/or milled to the desired particle size. The granulate may then be tabletted, or other excipients may be added prior to tabletting, such as a glidant and/or a lubricant.

A tabletting composition may be prepared conventionally by dry granulation. For example, the blended composition of the actives and excipients may be compacted into a slug or a sheet and then comminuted into compacted granules. The compacted granules may subsequently be compressed into a tablet.

As an alternative to dry granulation, a blended composition may be compressed directly into a compacted dosage form using direct compression techniques. Direct compression produces a uniform tablet without granules. Excipients that are particularly well suited for direct compression tabletting include microcrystalline cellulose, spray dried lactose, dicalcium phosphate dihydrate and colloidal silica. The proper use of these and other excipients in direct compression tabletting is known to those in the art with experience and skill in particular formulation challenges of direct compression tabletting.

A capsule filling of the present invention may comprise any of the aforementioned blends and granulates that were described with reference to tabletting, however, they are not subjected to a final tabletting step.

In further embodiments the composition of the invention may further comprise one or more additional active ingredients.

The details of the invention, its objects and advantages are explained hereunder in greater detail in relation to non-limiting exemplary illustrations.

### EXAMPLES

As used hereinafter in the examples, the term ‘1 volume’ means that for each gram of starting material 1 ml of solvent is used. The terms ‘2 volumes’, ‘3 volumes’ etc. are used accordingly.

### Example 1

### Preparation of N-phthaloyl-L-glutamine

To a suspension of phthalic anhydride (11.1 g or 0.076 mol) in dimethylformamide (DMF) (62 ml), L-glutamine (10 g or 0.067 mol) was added and the mixture was heated to a temperature of 90° C. to 95° C. for a period of 6 to 8 hours (or until completion of the reaction). When the reaction was complete, the excess solvent was removed by distillation at 65° C. to 70° C. under reduced pressure. The residue was cooled to a temperature of 25° C. to 30° C. and water (100 ml) was added. The solution was acidified with aqueous hydrochloric acid (50%) and stirred for a period of 8 to 10 hours. The resulting N-phthaloyl-L-glutamine was isolated by filtration, washed with water followed by methanol. The product was finally dried to a constant weight at a temperature of 55° C. to 60° C. under vacuum (80 to 100 mmHg) to produce an off-white solid.

Yield: 9.5 to 11 g (˜52.9% molar)

### Example 2

### Preparation of Thalidomide (α-form)

To a suspension of N-phthaloyl-L-glutamine (10 g or 0.036 mol) in acetonitrile (100 ml), carbonyl-diimidazole (7.65 g or 0.047 mol) and 4-dimethylaminopyridine (0.016 g or 1.3×10⁻³ mol) were added and the reaction mixture heated to a temperature of 75° C. to 77° C. and held at that temperature for a period of 6 to 8 hours (or until completion of the reaction). The reaction mixture was then allowed to cool to a temperature of 25° C. to 30° C. The solid product was then isolated by filtration and washed with methanol. Finally the product was dried to a constant weight under vacuum (˜100 mmHg) at a temperature of 50° C. to 55° C. to give the α-form of thalidomide as a white to off-white solid.

Yield: 6.5 to 7.0 g (˜70% molar) Melting Range: 271° C. to 274° C. HPLC purity: 99.89% (by area normalization) Polymorphic purity (as measured by XRPD): >99.5% DSC: Single peak at 274° C.

### Example 3

### Preparation of Thalidomide (α-form)

To a suspension of N-phthaloyl-L-glutamine (10 g or 0.036 mol) in acetone (100 ml), carbonyl-diimidazole (7.65 g or 0.047 mol) and 4-dimethylaminopyridine (0.016 g or 1.3×10⁻³ mol) were added and the reaction mixture heated to a temperature of 55° C. to 60° C. for a period of 6 to 8 hours (or until completion of the reaction). The reaction mixture was then allowed to cool to a temperature of 25° C. to 30° C. The solid product was then isolated by filtration and washed with methanol. Finally the product was dried to a constant weight under vacuum (˜100 mmHg) at a temperature of 50° C. to 55° C. to give the α-form of thalidomide as a white to off-white solid.

Yield: 6.0 to 6.5 g (˜65% molar) Melting Range: 271° C. to 274° C. HPLC purity: 99.85% (by area normalization) Polymorphic purity (as measured by XRPD): >99.5% DSC: Single peak at 273° C.

### Example 4

### Preparation of Thalidomide (α-form)

To a suspension of N-phthaloyl-L-glutamine (10 g or 0.036 mol) in a 1:1 (v/v) mixture of tetrahydrofuran (THF) and tertiary butyl methyl ether (TBME) (100 ml), carbonyl-diimidazole (7.65 g or 0.047 mol) and 4-dimethylaminopyridine (0.016 g or 1.3×10⁻³ mol) were added and the reaction mixture heated to a temperature of 65° C. to 70° C. for a period of 6 to 8 hours (or until completion of the reaction). The reaction mixture was then allowed to cool to a temperature of 25° C. to 30° C. The solid product was then isolated by filtration and washed with methanol. Finally the product was dried to a constant weight under vacuum (˜100 mmHg) at a temperature of 50° C. to 55° C. to give the α-form of thalidomide as a white to off-white solid.

Yield: 6.0 to 6.5 g (˜65% molar) Melting Range: 271° C. to 274° C. HPLC purity: 99.85% (by area normalization) Polymorphic purity (as measured by XRPD): >99.5% DSC: Single peak at 273° C.

### Example 5

### Chemical Purification of Thalidomide to Produce Thalidomide (α-form)

In order to further improve the chemical purity, the α-form of thalidomide (10 g), prepared by one of the methods outlined in examples 2 to 4, was dissolved in dimethylsulfoxide (DMSO) (50 ml or 5 volumes). This solution was slowly added, with stirring, to methanol (170 ml or 17 volumes) containing suspended seed crystals (1 to 5% w/w of input thalidomide) of α-form (prepared as per examples 2 to 4) at a temperature of 45° C. to 50° C. The mixture was then stirred for a further 30 to 50 minutes at a temperature of 45° C. to 50° C. The reaction mixture was then slowly cooled to a temperature of 35° C. to 40° C. and filtered. The solid was washed with methanol and vacuum filtered. Finally the solid pure product was dried to a constant weight under vacuum (˜100 mmHg) at a temperature of 50° C. to 55° C. to give the α-form of thalidomide as a white to off-white solid.

Yield: 8.0 to 8.5 g (˜85% w/w) Melting Range: 271° C. to 273° C. HPLC purity: 99.93% (by area normalization) Polymorphic purity (as measured by XRPD): >99.5% DSC: Single peak at 273.4° C.

### Example 6

### Preparation of Thalidomide (β-form)

To a suspension of N-phthaloyl-L-glutamine (10 g or 0.036 mol) in dimethylformamide (DMF) (60 ml), carbonyl-diimidazole (7.65 g or 0.047 mol) and 4-dimethylaminopyridine (0.016 g or 1.3×10⁻³ mol) were added and the reaction mixture heated to a temperature of 70° C. to 75° C. for a period of 7 to 8 hours (or until completion of the reaction). The heating was then stopped and the solvent was completely removed by distillation under reduced pressure. To the residual material, a 1:1 (v/v) mixture of methanol and water (90 ml or 9 volumes) was then added. The resulting solid was then filtered and washed with methanol. Finally the product was dried to a constant weight under vacuum (˜100 mmHg) at a temperature of 50° C. to 55° C. to give the β-form of thalidomide as a white to off-white solid.

Yield: 7.0 to 8.0 g (˜80% molar) Melting Range: 275° C. to 277° C. HPLC purity: 99.87% (by area normalization) Polymorphic purity (as measured by XRPD): >99.5% DSC: Single peak at 276.1° C.

### Example 7

### Chemical Purification of Thalidomide to Produce Thalidomide (β-form)

Thalidomide (either the α-form or the β-form or a mixture of α- and β-forms) (10 g) was dissolved in dimethylformamide (DMF) (60 ml or 6 volumes) and heated to a temperature of 70° C. to 75° C. for a period of 30 minutes to 2 hours. The solvent was then removed by distillation under reduced pressure (80 to 100 mmHg) at a temperature of 65° C. to 70° C. To the residual mass, acetone was added to produce a slurry which was stirred for 2 hours. The slurry was then filtered and washed with acetone. The solid was then dried to a constant weight under vacuum (80 to 100 mmHg) at a temp erature of 55° C. to 60° C. to give the β-form of thalidomide as a white to off-white solid.

Yield: 9.5 g (95% w/w) Melting Range: 275° C. to 277° C. HPLC purity: 99.89% (by area normalization) Polymorphic purity (as measured by XRPD): >99.5% DSC: Single peak at 276.4° C.

All of the thalidomide products produced by the above examples were found to have high polymorphic purity. The XRPD and DSC analyses showed no detectable levels of the β-form in the products of examples 2, 3, 4 and 5. The XRPD and DSC analyses also showed no detectable levels of the α-form in the products of examples 6 and 7.

It will be understood that the present invention has been described above by way of example only. The examples are not intended to limit the scope of the invention. Various modifications and embodiments can be made without departing from the scope and spirit of the invention, which is defined by the following claims only.

1-67. (canceled)

68. An anhydrous, crystalline α-form of thalidomide having a polymorphic purity greater than or equal to 95%.

69. An anhydrous, crystalline α-form of thalidomide according to claim 68:
(i) having a polymorphic purity greater than or equal to 97%; and/or
(ii) having a polymorphic purity greater than or equal to 99%; and/or
(iii) having a polymorphic purity greater than or equal to 99.5%; and/or
(iv) having a polymorphic purity greater than or equal to 99.9%; and/or
(v) having a chemical purity greater than or equal to 99%; and/or
(vi) having a chemical purity greater than or equal to 99.5%; and/or
(vii) having a chemical purity greater than or equal to 99.8%; and/or
(viii) containing less than or equal to 5% of crystalline β-form of thalidomide; and/or
(ix) containing less than or equal to 3% of crystalline β-form of thalidomide; and/or
(x) containing less than or equal to 1% of crystalline β-form of thalidomide; and/or
(xi) containing less than or equal to 0.5% of crystalline β-form of thalidomide; and/or
(xii) containing less than or equal to 0.1% of crystalline β-form of thalidomide.

70. An anhydrous, crystalline α-form of thalidomide having a chemical purity greater than or equal to 99%.

71. An anhydrous, crystalline α-form of thalidomide according to claim 70:
(i) having a chemical purity greater than or equal to 99.5%; and/or
(ii) having a chemical purity greater than or equal to 99.8%; and/or
(iii) having a polymorphic purity greater than or equal to 95%; and/or
(iv) having a polymorphic purity greater than or equal to 97%; and/or
(v) having a polymorphic purity greater than or equal to 99%; and/or
(vi) having a polymorphic purity greater than or equal to 99.5%; and/or
(vii) having a polymorphic purity greater than or equal to 99.9%; and/or
(viii) containing less than or equal to 5% of crystalline β-form of thalidomide; and/or
(ix) containing less than or equal to 3% of crystalline β-form of thalidomide; and/or
(x) containing less than or equal to 1% of crystalline β-form of thalidomide; and/or
(xi) containing less than or equal to 0.5% of crystalline β-form of thalidomide; and/or
(xii) containing less than or equal to 0.1% of crystalline β-form of thalidomide.

72. An anhydrous, crystalline β-form of thalidomide having a polymorphic purity greater than or equal to 95%.

73. An anhydrous, crystalline β-form of thalidomide according to claim 72:
(i) having a polymorphic purity greater than or equal to 97%; and/or
(ii) having a polymorphic purity greater than or equal to 99%; and/or
(iii) having a polymorphic purity greater than or equal to 99.5%; and/or
(iv) having a polymorphic purity greater than or equal to 99.9%; and/or
(v) having a chemical purity greater than or equal to 99%; and/or
(vi) having a chemical purity greater than or equal to 99.5%; and/or
(vii) having a chemical purity greater than or equal to 99.8%; and/or
(viii) containing less than or equal to 5% of crystalline α-form of thalidomide; and/or
(ix) containing less than or equal to 3% of crystalline α-form of thalidomide; and/or
(x) containing less than or equal to 1% of crystalline α-form of thalidomide; and/or
(xi) containing less than or equal to 0.5% of crystalline α-form of thalidomide; and/or
(xii) containing less than or equal to 0.1% of crystalline α-form of thalidomide.

74. An anhydrous, crystalline β-form of thalidomide having a chemical purity greater than or equal to 99%.

75. An anhydrous, crystalline β-form of thalidomide according to claim 74:
(i) having a chemical purity greater than or equal to 99.5%; and/or
(ii) having a chemical purity greater than or equal to 99.8%; and/or
(iii) having a polymorphic purity greater than or equal to 95%; and/or
(iv) having a polymorphic purity greater than or equal to 97%; and/or
(v) having a polymorphic purity greater than or equal to 99%; and/or
(vi) having a polymorphic purity greater than or equal to 99.5%; and/or
(vii) having a polymorphic purity greater than or equal to 99.9%; and/or
(viii) containing less than or equal to 5% of crystalline α-form of thalidomide; and/or
(ix) containing less than or equal to 3% of crystalline α-form of thalidomide; and/or
(x) containing less than or equal to 1% of crystalline α-form of thalidomide; and/or
(xi) containing less than or equal to 0.5% of crystalline α-form of thalidomide; and/or
(xii) containing less than or equal to 0.1% of crystalline α-form of thalidomide.

76. An anhydrous, crystalline α-form of thalidomide containing less than or equal to 5% of crystalline β-form of thalidomide.

77. An anhydrous, crystalline α-form of thalidomide according to claim 76:
(i) containing less than or equal to 3% of crystalline β-form of thalidomide; and/or
(ii) containing less than or equal to 1% of crystalline β-form of thalidomide; and/or
(iii) containing less than or equal to 0.5% of crystalline β-form of thalidomide; and/or
(iv) containing less than or equal to 0.1% of crystalline β-form of thalidomide; and/or
(v) having a chemical purity greater than or equal to 99%; and/or
(vi) having a chemical purity greater than or equal to 99.5%; and/or
(vii) having a chemical purity greater than or equal to 99.8%; and/or
(viii) having a polymorphic purity greater than or equal to 95%; and/or
(ix) having a polymorphic purity greater than or equal to 97%; and/or
(x) having a polymorphic purity greater than or equal to 99%; and/or
(xi) having a polymorphic purity greater than or equal to 99.5%; and/or
(xii) having a polymorphic purity greater than or equal to 99.9%.

78. An anhydrous, crystalline β-form of thalidomide containing less than or equal to 5% of crystalline α-form of thalidomide.

79. An anhydrous, crystalline β-form of thalidomide according to claim 78:
containing less than or equal to 3% of crystalline α-form of thalidomide; and/or
(ii) containing less than or equal to 1% of crystalline α-form of thalidomide; and/or
(iii) containing less than or equal to 0.5% of crystalline α-form of thalidomide; and/or
(iv) containing less than or equal to 0.1% of crystalline α-form of thalidomide; and/or
(v) having a chemical purity greater than or equal to 99%; and/or
(vi) having a chemical purity greater than or equal to 99.5%; and/or
(vii) having a chemical purity greater than or equal to 99.8%; and/or
(viii) having a polymorphic purity greater than or equal to 95%; and/or
(ix) having a polymorphic purity greater than or equal to 97%; and/or
(x) having a polymorphic purity greater than or equal to 99%; and/or
(xi) having a polymorphic purity greater than or equal to 99.5%; and/or
(xii) having a polymorphic purity greater than or equal to 99.9%.

80. A process for preparing an anhydrous, crystalline α-form of thalidomide, comprising cyclizing N-phthaloyl-glutamine in an organic solvent system and isolating the anhydrous, crystalline α-form of thalidomide.

81. A process according to claim 80, wherein:
(i) N-phthaloyl-glutamine is cyclized by reaction with a coupling agent; and/or
(ii) N-phthaloyl-glutamine is cyclized by reaction with a coupling agent, wherein the coupling agent is selected from the group consisting of carbonyl diimidazole (CDI), phosphorus oxychloride, thionyl chloride, urea, thiourea, acid chloride, acetic anhydride, phosgene, ethyl chloroformate, thionyl diimidazole, pivaloyl chloride, tosyl chloride, mesyl chloride, tosyl imidazole, 1-ethyl-3-(3-dimethylaminopropyl) carbodiimide (EDCI), 2-chloro-N-methyl-pyridinium iodide, 2-(1H-benzotriazol-1-yl)-1,1,3,3-tetramethyluronium hexafluorophosphate (HBTU) and 2-(benzotriazol-1-yl)oxytris(dimethylamino)phosphonium hexafluorophosphate (BOP) or mixtures thereof; and/or
(iii) N-phthaloyl-glutamine is cyclized by reaction with a coupling agent, wherein the coupling agent is carbonyl diimidazole (CDI); and/or
(iv) N-phthaloyl-glutamine is cyclized in the presence of a catalyst; and/or
(v) N-phthaloyl-glutamine is cyclized in the presence of a catalyst, wherein the catalyst is selected from the group consisting of 4-dimethylaminopyridine (DMAP), pyridine, diethylaminopyridine, 1,8-diazabicyclo[5,4,0]undec-7-ene (DBU), 1,4-diazabicyclo[2,2,2]octane (DABCO) and 1,5-diazabicyclo[4,3,0]non-5-ene (DBN) or mixtures thereof; and/or
(vi) N-phthaloyl-glutamine is cyclized in the presence of a catalyst, wherein the catalyst is 4-dimethylaminopyridine (DMAP); and/or
(vii) the organic solvent system comprises a solvent selected from the group comprising straight chain or branched aliphatic ketones, aliphatic nitriles, ethers and mixtures thereof; and/or
(viii) the organic solvent system comprises a solvent selected from the group consisting of acetone, butanone or mixtures thereof; and/or
(ix) the organic solvent system comprises acetone; and/or
(x) the organic solvent system comprises a solvent selected from the group consisting of acetonitrile, propionitrile or mixtures thereof; and/or
(xi) the organic solvent system comprises acetonitrile; and/or
(xii) the organic solvent system comprises a solvent selected from the group consisting of tetrahydrofuran (THF), tertiary butyl methyl ether (TBME) or mixtures thereof; and/or
(xiii) the organic solvent system comprises a mixture of tetrahydrofuran (THF) and tertiary butyl methyl ether (TBME); and/or
(xiv) the reaction mixture is heated to a temperature between about 50° C. and about 100° C.; and/or
(xv) the reaction mixture is heated to a temperature between about 50° C. and about 77° C.; and/or
(xvi) the reaction mixture is heated to a temperature between about 50° C. and about 100° C., and wherein the reaction mixture is further cooled in order to isolate the anhydrous, crystalline α-form of thalidomide; and/or
(xvii) the reaction mixture is heated to a temperature between about 50° C. and about 77° C., and wherein the reaction mixture is further cooled in order to isolate the anhydrous, crystalline α-form of thalidomide.

82. A process for preparing an anhydrous, crystalline β-form of thalidomide, comprising cyclizing N-phthaloyl-glutamine in an organic solvent system, heating the reaction mixture and isolating the anhydrous, crystalline β-form of thalidomide.

83. A process according to claim 82, wherein:
(i) N-phthaloyl-glutamine is cyclized by reaction with a coupling agent; and/or
(ii) N-phthaloyl-glutamine is cyclized by reaction with a coupling agent, wherein the coupling agent is selected from the group consisting of carbonyl diimidazole (CDI), phosphorus oxychloride, thionyl chloride, urea, thiourea, acid chloride, acetic anhydride, phosgene, ethyl chloroformate, thionyl diimidazole, pivaloyl chloride, tosyl chloride, mesyl chloride, tosyl imidazole, 1-ethyl-3-(3-dimethylaminopropyl) carbodiimide (EDCI), 2-chloro-N-methyl-pyridinium iodide, 2-(1H-benzotriazol-1-yl)-1,1,3,3-tetramethyluronium hexafluorophosphate (HBTU) and 2-(benzotriazol-1-yl)oxytris(dimethylamino)phosphonium hexafluorophosphate (BOP) or mixtures thereof; and/or
(iii) N-phthaloyl-glutamine is cyclized by reaction with a coupling agent, wherein the coupling agent is carbonyl diimidazole (CDI); and/or
(iv) N-phthaloyl-glutamine is cyclized in the presence of a catalyst; and/or
(v) N-phthaloyl-glutamine is cyclized in the presence of a catalyst, wherein the catalyst is selected from the group consisting of 4-dimethylaminopyridine (DMAP), pyridine, diethylaminopyridine, 1,8-diazabicyclo[5,4,0]undec-7-ene (DBU), 1,4-diazabicyclo[2,2,2]octane (DABCO) and 1,5-diazabicyclo[4,3,0]non-5-ene (DBN) or mixtures thereof; and/or
(vi) N-phthaloyl-glutamine is cyclized in the presence of a catalyst, wherein the catalyst is 4-dimethylaminopyridine (DMAP); and/or
(vii) the organic solvent system comprises solvents selected from the group comprising dimethylformamide (DMF) and dimethylacetamide or mixtures thereof; and/or
(viii) the organic solvent system comprises dimethylformamide (DMF); and/or
(ix) the reaction mixture is heated to a temperature between about 50° C. and about 100° C.; and/or
(x) the reaction mixture is heated to a temperature between about 70° C. and about 75° C.; and/or
(xi) isolating the anhydrous, crystalline β-form of thalidomide comprises removal of the organic solvent system, addition of a second solvent, and isolating the anhydrous, crystalline β-form of thalidomide; and/or
(xii) isolating the anhydrous, crystalline β-form of thalidomide comprises removal of the organic solvent system, addition of a second solvent, and isolating the anhydrous, crystalline β-form of thalidomide, wherein the second solvent is selected from the group consisting of methanol, water, acetone or mixtures thereof; and/or
(xiii) isolating the anhydrous, crystalline β-form of thalidomide comprises removal of the organic solvent system, addition of a second solvent, and isolating the anhydrous, crystalline β-form of thalidomide, wherein the second solvent is acetone; and/or
(xiv) isolating the anhydrous, crystalline β-form of thalidomide comprises removal of the organic solvent system, addition of a second solvent, and isolating the anhydrous, crystalline β-form of thalidomide, wherein the second solvent is a mixture of methanol and water.

84. A process for preparing a pure, anhydrous, crystalline α-form of thalidomide, comprising dissolving thalidomide in dimethylsulfoxide (DMSO), adding the mixture to methanol containing suspended seed crystals of the α-form of thalidomide, and isolating the pure, anhydrous, crystalline α-form of thalidomide.

85. A process according to claim 84, wherein:
(i) the thalidomide starting material is selected from the group consisting of crystalline α-form of thalidomide and a mixture of α-form and β-form; and/or
(ii) the reaction mixture is heated to a temperature between about 40° C. and about 50° C.; and/or
(iii) the reaction mixture is cooled in order to isolate the pure, anhydrous, crystalline α-form of thalidomide; and/or
(iv) the reaction mixture is cooled to a temperature between about 30° C. to about 40° C. in order to isolate the pure, anhydrous, crystalline α-form of thalidomide.

86. A pure, anhydrous, crystalline α-form of thalidomide having a chemical purity greater than or equal to 99.9%, prepared by a process according to claim 84.

87. A process for preparing a pure, anhydrous, crystalline β-form of thalidomide, comprising dissolving thalidomide in dimethylformamide (DMF), heating the reaction mixture, and isolating the pure, anhydrous, crystalline β-form of thalidomide.

88. A process according to claim 87, wherein:
(i) the thalidomide starting material is selected from the group consisting of crystalline α-form of thalidomide, crystalline β-form of thalidomide and a mixture of α-form and β-form; and/or
(ii) the reaction mixture is heated to a temperature between about 50° C. and about 100° C.; and/or
(iii) the reaction mixture is heated to a temperature between about 70° C. and about 75° C.; and/or
(iv) isolating the pure, anhydrous, crystalline β-form of thalidomide comprises removal of DMF, addition of a second solvent, and isolating the pure, anhydrous, crystalline β-form of thalidomide; and/or
(v) isolating the pure, anhydrous, crystalline β-form of thalidomide comprises removal of DMF, addition of a second solvent, and isolating the pure, anhydrous, crystalline β-form of thalidomide, wherein the second solvent is selected from the group consisting of methanol, water, acetone or mixtures thereof; and/or
(vi) isolating the pure, anhydrous, crystalline β-form of thalidomide comprises removal of DMF, addition of a second solvent, and isolating the pure, anhydrous, crystalline β-form of thalidomide, wherein the second solvent is acetone; and/or
(vii) isolating the pure, anhydrous, crystalline β-form of thalidomide comprises removal of DMF, addition of a second solvent, and isolating the pure, anhydrous, crystalline β-form of thalidomide, wherein the second solvent is a mixture of methanol and water.

89. A pure, anhydrous, crystalline β-form of thalidomide having a chemical purity greater than or equal to 99.9%, prepared by a process according to claim 87.

90. A pharmaceutical composition comprising an anhydrous, crystalline α-form of thalidomide according to claim 68 and one or more pharmaceutically acceptable excipients.

91. A pharmaceutical composition comprising an anhydrous, crystalline α-form of thalidomide according to claim 70 and one or more pharmaceutically acceptable excipients.

92. A pharmaceutical composition comprising an anhydrous, crystalline α-form of thalidomide according to claim 76 and one or more pharmaceutically acceptable excipients.

93. A pharmaceutical composition comprising an anhydrous, crystalline β-form of thalidomide according to claim 72 and one or more pharmaceutically acceptable excipients.

94. A pharmaceutical composition comprising an anhydrous, crystalline β-form of thalidomide according to claim 74 and one or more pharmaceutically acceptable excipients.

95. A pharmaceutical composition comprising an anhydrous, crystalline β-form of thalidomide according to claim 78 and one or more pharmaceutically acceptable excipients.

96. A method of treating erythema nodosum leprosum (ENL) or multiple myeloma, comprising administering to a patient in need thereof a therapeutically effective amount of an anhydrous, crystalline α-form of thalidomide according to claim 68.

97. A method of treating erythema nodosum leprosum (ENL) or multiple myeloma, comprising administering to a patient in need thereof a therapeutically effective amount of an anhydrous, crystalline α-form of thalidomide according to claim 70.

98. A method of treating erythema nodosum leprosum (ENL) or multiple myeloma, comprising administering to a patient in need thereof a therapeutically effective amount of an anhydrous, crystalline α-form of thalidomide according to claim 76.

99. A method of treating erythema nodosum leprosum (ENL) or multiple myeloma, comprising administering to a patient in need thereof a therapeutically effective amount of an anhydrous, crystalline β-form of thalidomide according to claim 72.

100. A method of treating erythema nodosum leprosum (ENL) or multiple myeloma, comprising administering to a patient in need thereof a therapeutically effective amount of an anhydrous, crystalline β-form of thalidomide according to claim 74.

101. A method of treating erythema nodosum leprosum (ENL) or multiple myeloma, comprising administering to a patient in need thereof a therapeutically effective amount of an anhydrous, crystalline β-form of thalidomide according to claim 78.
